(19) United States
(12) Patent Application Publication
(10) Pub. No.: US 2020/0360918 A1
(43) Pub. Date: Nov. 19, 2020

(54) MULTIPIN SOLID PHASE MICROEXTRACTION DEVICE

(71) Applicants: Sigma-Aldrich Co. LLC, St. Louis, MO (US); EMD Millipore Corporation, Burlington, MA (US)

(72) Inventors: Yong Chen, Mississauga (CA); Olga I. Shimelis, State College, PA (US); Dave Bell, Bellefonte, PA (US); Katherine K. Stenerson, State College, PA (US); Craig Aurund, Lock Haven, PA (US); Emily Barrey, (US); Xiaoning Lu, State College, PA (US); Kurt Greenizen, Atkinson, NH (US)

(21) Appl. No.: 16/638,845

(22) PCT Filed: Aug. 14, 2018
(86) PCT No.: PCT/US2018/046613

§ 371 Date: Feb. 13, 2020

Related U.S. Application Data
(60) Provisional application No. 62/545,138, filed on Aug. 14, 2017.

(51) Int. Cl. B01L 3/02; B01L 3/00; G01N 35/10; G01N 1/40; G01N 33/94

(52) U.S. Cl. CPC B01L 3/0244; B01L 3/5025; G01N 35/1095; G01N 35/1009; G01N 1/405; B01L 2300/163; G01N 2035/1037; B01L 2200/025; B01L 2200/0631; B01L 2300/0829; G01N 33/94

(57) ABSTRACT

An apparatus for simultaneously extracting one or more analytes from a plurality of samples, the apparatus comprising a housing having an upper platform having a plurality of pins, each pin suitable for BioSPME, and a lower platform having a plurality of wells for sample solutions, such when the upper platform is fitted over the lower platform, the pins of the upper platform fit into the wells of the lower platform such that the pins are immersed into the samples, allowing for simultaneous extraction of analytes in each sample. The apparatus can be coupled with other instruments to enable measurement of total and free analytes in each sample. Also provided are methods of simultaneous extraction of analytes in a plurality of samples using the apparatus described herein.

### CROSS-REFERENCE TO RELATED APPLICATIONS

This application claims the benefit of priority of U.S. Provisional Patent Application No. 62/545,138 filed Aug. 14, 2017, the entirety of which is incorporated herein by reference.

### BACKGROUND OF THE INVENTION

Matrix compatible solid phase micro extraction, or BioSPME, is an important analytical technique for rapid and accurate determination of analytes of interest in complicated samples. BioSPME has proven especially useful in testing of biological samples, such as blood, plasma, urine, saliva, tissue, and food products.

BioSPME offers several advantages over other analytical methods including: method simplicity—single-step sampling and sample preparation combined reduces cost and time; direct sampling—no sample pretreatment is necessary; selectivity—allows for direct measurement of free analyte fraction in complicated samples; small sample volumes—allows for analysis of small and precious samples; small desorption volumes—increased sample concentration enables detection of less prevalent analytes in samples.

Automation is highly desired both for high throughput as well as decreased human error. While advances in the materials used to produce fibers has allowed the BioSPME to become more automation friendly, there are no BioSPME devices currently available that allow automated determination of both total and free analytes with in a set of samples. Accordingly, a need exists for new BioSPME devices allowing for better automation.

### SUMMARY OF THE INVENTION

Provided herein are apparatuses for simultaneous extraction of one or more analytes from a plurality of samples; the apparatus includes a housing having an upper platform and a lower platform wherein the upper platform and the lower platform are separate pieces that fit together; the upper platform having a top surface and a bottom surface, the bottom surface including a plurality of pins integral thereto perpendicular to the bottom surface, the pins having a surface adapted to solid phase microextraction (SPME); the lower platform having a top surface and a bottom surface, the top surface comprising a plurality of individual wells. When the upper platform and the lower platform are joined together, at least one pin of the upper platform is disposed into at least one well of the lower platform. In a preferred embodiment, the when the upper platform and the lower platform are joined together, each pin on the upper platform is disposed into a single well on the lower platform.

In a preferred embodiment, the pins are rod-like in shape, preferably, cylindrical or frustoconical; however, in other embodiments, the pins may be conical, rectangular, and so forth. The pins are adapted for solid phase microextraction, by, for example, coating or otherwise incorporating into the pins a stationary phase capable of absorbing or adsorbing analytes of interest. Preferably, the pins are adapted to be useful for BioSPME. In a preferred embodiment, the pins have a coating including microspheres and a binder. In various embodiments, the coating includes functionalized silica spheres, functionalized carbon spheres, polymeric resins, and combinations thereof in a biocompatible polymeric binder such as polyacrylonitrile (PAN), polyethylene glycol (PEG), polypyrrole, derivatized cellulose, polysulfone, polyacrylamide, or polyamide. In a particularly preferred embodiment, the pins are coated with functionalized silica microparticles in a polyacrylonitrile (PAN) binder.

The upper platform includes a plurality of pins perpendicular to the bottom surface of the platform, which form the substrates for extracting analytes from samples. The pins may be made of any material useful as a substrate for SPME, including but not limited to polymers, silica, and metals. Unlike conventional SPME substrates, such as fibers or metal blades, the apparatus described herein includes a plurality of pins arranged in a manner as to be conducive to use with automated sample handling systems and robot sampling systems. The pins may be formed by known methods and incorporated into the upper platform in a separate step, or the pins may be formed at the same time as the upper platform, such as by injection molding or 3-d printing. In a preferred embodiment, the pins are polymeric. In particularly preferred embodiment, the pins are made of polyethylene or polypropylene.

Preferably, the pins have a diameter in the range from about 0.2 mm to about 5 mm. In preferred embodiments, the diameter of the pins is in the range from about 0.5 mm to about 2 mm. In a particularly preferred embodiment, the pins have a diameter of about 1 mm. The length of the pin can be varied, as for example, to accommodate various sample volumes and well depths. The length of the pins is preferably in the range from about 0.2 mm to about 5 cm. In some embodiments, the length may be from about 0.5 mm to about 2.5 cm. In other embodiments, the length may be from about 1 mm to about 1 cm. Moreover, suitable lengths can be determined based on the matching well size in the lower platform, desired sample volumes, and other such considerations. It is recognized that the depth, diameter, and volume of the well in the lower plate can also be varied.

In some embodiments, the microspheres and the binder may be integrated into the pins when the pins are formed. For example, the pins may be formed by 3-d printing, incorporating the binder and the microspheres directly into the ink matrix used to form the pins. The printed pins are then ready to use without the need for a separate coating step.

In preferred embodiments, the pins are adapted to measuring analytes in biological samples, such as such as blood, plasma, urine, saliva, tissue, and food products through the use of the microspheres and binder as described herein.

In a preferred embodiment, the apparatuses described herein are arranged such that the pins and the wells are compatible with conventional multiwell platforms, including, but not limited to 96-well platforms, 384-well platforms, and 1534-well platforms. The upper platform may be configured to have the same number of pins as the number of wells in the lower platform, or the upper platform may be configured to have a different number of pins from the number of wells in the lower platform.

In a particularly preferred embodiment, the apparatuses described herein are adapted to interface with an automated liquid handling system. In preferred embodiments, the apparatuses are adapted to interface with an automated liquid handling system that includes an interface to a mass spectrometer. The apparatuses described herein are particularly well-suited to interface with mass spectrometers that have an ionization source such as electrospray, Desorption Electrospray Ionization (DESI), and Direct Analysis in Real Time (DART).

Also provided is a method of simultaneously isolating free analytes from a plurality of samples using the apparatuses described herein. The method is performed by adding a plurality of samples containing free analytes into the wells of the lower platform; joining the upper platform with the lower platform, wherein the pins on the upper platform are disposed in the wells of the lower platform such that the pins are in contact with the samples; and maintaining the pins in contact with the samples for sufficient time for the free analytes to be extracted.

In a preferred embodiment, the apparatus coupled with an automatic liquid handling system, and preferably interfaced with a mass spectrometer. In such methods, apparatus can be used in the automated system to provide both the total and free analytes in the each sample.

### BRIEF DESCRIPTION OF THE DRAWINGS

FIG. 1 illustrates (A) a small volume 96-pin upper platform with (B) accompanying sample reservoir lower platform.

FIG. 2 illustrates the interface of the 96-pin upper platform with the sample reservoir/lower platform.

FIG. 3 illustrates (A) a commercially available LC tip BioSPME device having a coated fiber housed within a pipette tip; (B) is a schematic of an extraction performed with a BioSPME fiber.

FIG. 4 illustrates (A) 96-pin and (B) 384-pin embodiments of the SPME devices as described herein.

FIG. 5 (A) illustrates an 8-pin strip device and 96-well sample reservoir adapted for use with the 8-pin strip; (B) further illustrates the cover on the sample reservoir.

FIG. 6 (A) illustrates a 12-pin strip device and 96-well sample reservoir adapted for use with the 12-pin strip; (B) further illustrates the cover on the sample reservoir.

FIG. 7 (A) illustrates the coating on the ends of the substrate pins; (B) illustrates only the coated tip closer up; (C) and (D) shows the actual coated substrate pins at low and high magnification.

FIG. 8 illustrates the interface of a coated pin device with an automated liquid handling system; (A) shows the interface of the upper platform with the automated sample handling system and (B) shows the interface of the lower platform with the sample handling system.

FIG. 9 illustrates a coated pin device interfacing with samples in a 96-well configuration; (A) illustrates the upper and lower platforms when the pins are not disposed in the sample wells, as before or after an extraction; (B) illustrates the pins disposed in the sample wells.

### DETAILED DESCRIPTION OF THE INVENTION

The apparatuses provided herein allows for an automated platform for the determination of free and total analyte from a variety of matrices, including biological samples, while being adaptable to a wide range of liquid handling systems and robotic sampling instrumentation. These devices use minimal sample size and because they are automatable, allow for quicker analyses, with less potential for error.

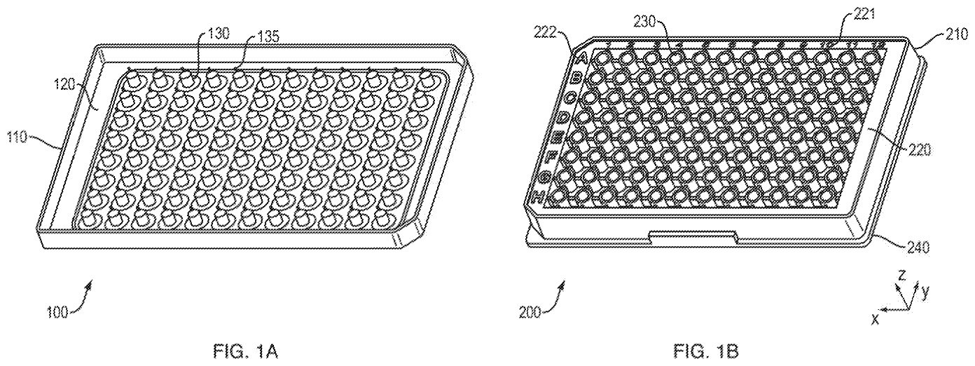

The apparatuses provided herein conveniently allow for simultaneous extraction of one or more analytes from a plurality of samples, up to one sample per pin/well combination. FIG. 1A shows an apparatus as described herein with the upper platform, 100, having a surface 120 with 96-pins, 130, in an eight pin-by-twelve pin arrangement. Each pin is illustrated as having a tip 135 opposite the surface; the tip including a BioSPME coating. In the embodiment illustrated, the upper platform includes a lip 110 around the perimeter, which fits around the perimeter of the lower platform 200 when the upper platform is engaged or removably joined with the lower platform in the closed or sampling position.

FIG. 1B illustrates the lower platform, 200, including 96 separate reservoirs or wells, 230, arranged within top surface 220. The wells 230 are configured in an eight pin-by-twelve pin arrangement to align in a one pin-to-one well arrangement with the pins 130 of the upper platform 200 when the upper and lower platforms are joined. As illustrated, the top surface includes numbers 221 along one edge in the x-direction and letters 222 along one edge in the y-direction, to reference specific wells in the array. The outer edge 210 of the lower platform 200 as illustrated, i.e., the z-direction, fits within lip 110 of the upper platform when in the closed or sampling position. Lower platform 200 also includes a flange 240 around the bottom.

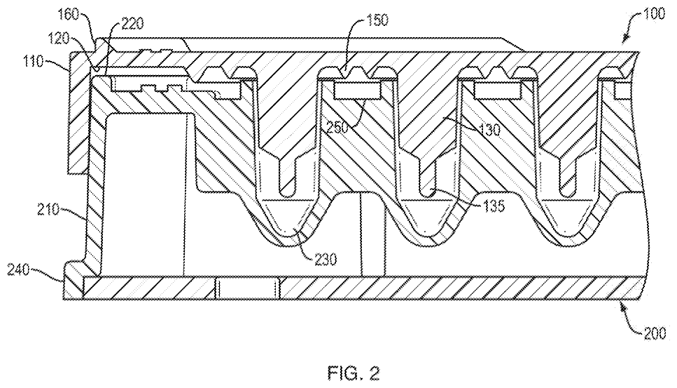

The interface between the upper platform and the lower platform is shown in FIG. 2, with shows the housing with the upper platform 100 and lower platform 200 in a closed or sampling position. As illustrated, upper platform 100 includes lip 100 that fits over the lower platform when the upper and lower platform are removably joined, as during sampling, or during shipping or storage. Pins 130 are shown as integral to the inner surface 120 in this embodiment. The pins, 130, which have a matrix compatible BioSPME coating on each tip, 135, fit into wells 230 of lower platform 200 and contact samples within the wells wherein analytes of interest are extracted by the coated pins. The upper platform may also include inner positioning members 150 and outer positioning members 160. The inner positioning members fit into receiving channels 250 of the lower platform. The outer positioning members 160 of upper platform 100 and flange 240 of lower platform 200 may be used, e.g., in interfacing with autosamplers.

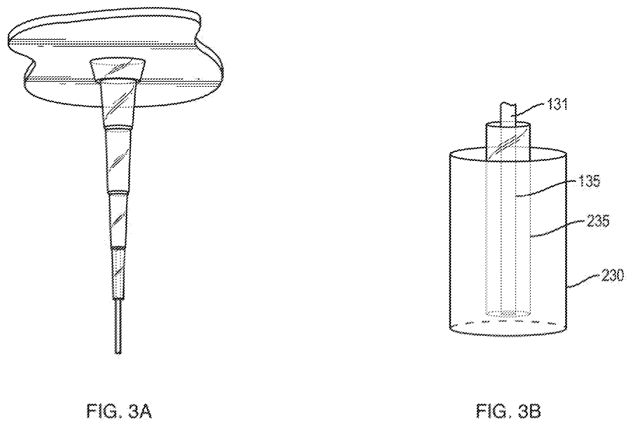

Matrix compatible solid phase micro extraction (BioSPME) utilizes functionalized particles that are embedded onto the surface of a core substrate, such as a fiber, using a polymeric binder. A commercially available BioSPME device is illustrated in FIG. 3. It includes a single fiber 131 as the supporting core substrate with a coating 135 that includes functionalized particles embedded in an inert binder. The fiber 131 is housed within a pipette tip, as shown in FIG. 3A. FIG. 3B illustrates the use of the coated fiber in a typical SPME process. The coated fiber contacts a sample 235, such as blood, plasma, serum, urine, saliva, and so forth, within a sample well 230. The extracted components are analyzed using conventional methods.

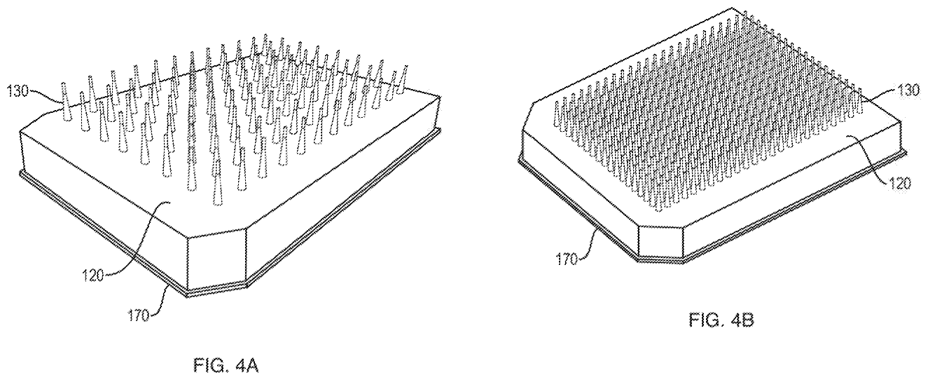

A notable feature of the devices described herein is that they differ from conventional SPME or BioSPME devices in that the core substrates are, rather than the conventional fibers, blades, or mesh, a plurality of pins that are integral to a surface, such as a platform, as shown for example, in FIG. 4. The pins may be arranged in virtually any arrangement, but preferably, the pins are arranged such that they can be disposed into the wells of a conventional well platform. In some embodiments, the pins are arranged in strips, in other embodiments, the pins are arranged in arrays. Other arrangements, however, are also possible and could be configured by one skilled in the art.

Another embodiment of upper platform 100 illustrated in FIG. 4, which shows 96-pin and 384-pin embodiments of upper platform 100. The pins 130 are integral to surface 120. As illustrated flange 170 on the outer perimeter can be used to interface with a upper portion of a housing, as illustrated in FIG. 1, or alternatively, can be used to interface directly with an autosampler. In a particular embodiment, the multipin SPME device may be a plate having a top surface and a bottom surface, wherein the bottom surface has pins that are configured to fit into or match the wells of a conventional multiwell plate. That is, the multipin SPME device and the multiwell plate of this embodiment may, but do not have to, form a single housing when coupled for use, i.e., performing extractions. In this embodiment, the bottom surface includes a plurality of pins, each pin integral to the bottom surface, either formed as a single piece as by injection molding or 3-d printing, or otherwise fixed to the bottom surface such as by fixing metal, silica or polymeric pins to a polymeric plate by known methods, and projecting outwards in an approximately perpendicular manner to an end opposite the bottom surface. The pins have a surface adapted to SPME, preferably BioSPME, typically through a coating, such as functionalized silica spheres in a biocompatible binder, a polymeric coating, etc. The coated pins are configured to fit into a plurality of wells in a conventional multiwell platform such that the surface adapted to SPME can contact a sample disposed in one or more wells in the well plate. The multipin device may be configured for any conventional multiwell platform, such as for example, 8-well plates or strips, 96-well plates, 384-well plates, 1534-well plates, and so forth.

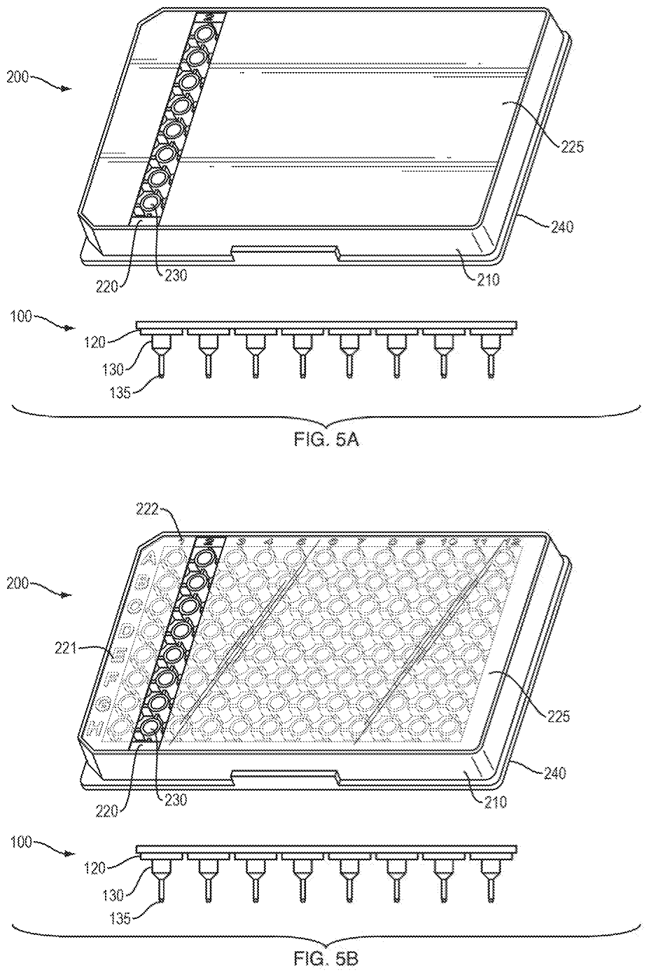

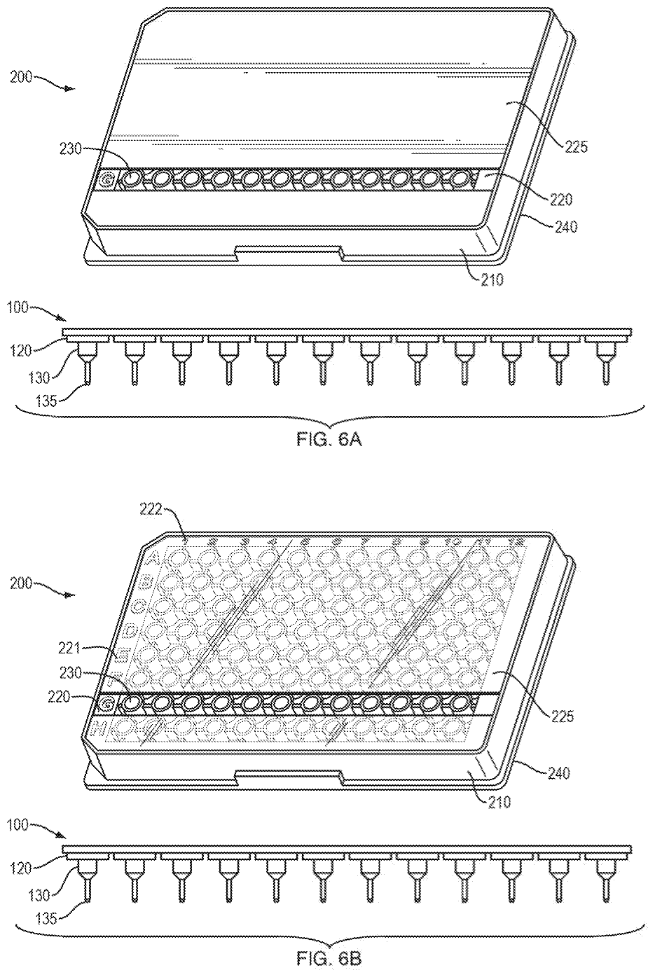

In certain embodiments, the upper platform may have fewer pins than the number of wells in the lower platform. FIGS. 5 and 6 show two exemplary, non-limiting, embodiments based on a 96-well bottom platform configured as eight rows of wells by twelve rows of wells. In FIG. 5, the upper platform includes eight pins and is configured to fit in one 8-well row of the bottom platform. In FIG. 6, the upper platform includes twelve pins and is configured to fit in one 12-well row of the bottom platform. In such embodiments, a plate 225 may be used to cover the extra wells in the lower platform, however, it is noted that such a plate is not necessary for the operation of the apparatus. Alternately, these configurations can be achieved by including only 8 or 12 pins, in the same configuration, in an upper platform that is the same size as the lower platform. These configurations are illustrative of two possible configurations. One skilled in the art will recognize other configurations are possible, provided the one or more pins of the upper platform can be disposed into one or more wells of the lower platform.

The reservoirs or wells of the apparatuses described herein may be of any conventional well shape, including, e.g., flat bottom, round bottom, V-bottom or conical bottom. In some alternate embodiments, the reservoirs or wells may be substantially larger than the pins, allowing more than one pin to fit into a single well. While this configuration may obviate some advantages of the smaller well size, such as sample size, it may have other advantages, such as the ability to simultaneously use pins with different coatings, providing the ability to extract different classes of analytes simultaneously.

In a preferred embodiment, the pins are cylindrical in shape; however, in other embodiments, the pins may be rod-like, frustoconical, conical, rectangular, and so forth. The pins are adapted to be useful for BioSPME. In particular, the pins include a combination of microparticles and a biocompatible polymeric binder to enable efficient isolation of target analytes from sample matrix with minimum amount of required sample.

In various embodiments, the coating includes microparticles, or microspheres, such as functionalized silica spheres, functionalized carbon spheres, polymeric resins, and combinations thereof. Typically, microspheres useful for liquid chromatography, i.e., affinity chromatography, as well as those useful for solid phase extraction (SPE) and solid phase micro extraction (SPME) are preferred for the coatings on the pins of the described apparatus.

In particular, the microspheres may include functionalized silica microspheres, such as, for example, C-18/silica (silica particles derivatized with a hydrophobic phase containing octadecyl), RP-amide-silica (silica having a bonded phase containing palmitamidopropyl), or HS-F5-silica (silica with a bonded phase containing pentafluorophenyl-propyl).

Some other non-limiting examples of suitable microparticles include: normal-phase silica, C1/silica, C4/silica, C6/silica, C8/silica, C18/silica, C30/silica, phenyl/silica, cyano/silica, diol/silica, ionic liquid/silica, Titan™ silica (MilliporeSigma), molecular imprinted polymer microparticles, hydrophilic-lipophilic-balanced (HLB) microparticles, Carboxen® 1006 (MilliporeSigma) or divinylbenzene. Mixtures of microparticles can also be used in the coatings. The microspheres used in the coatings for the pins may be inorganic (e.g. silica), organic (e.g. Carboxen® or divinylbenzene) or inorganic/organic hybrid (e.g. silica and organic polymer). In a preferred embodiment, the microspheres are C18/silica.

The particles, or microspheres, may have diameters in the range from about 10 nm to about 1 mm. In some embodiments, the spherical particles have diameters in the range from about 20 μm to about 125 μm. In certain embodiments, the microspheres have a diameter in the range from about 30 μm to about 85 μm. In some embodiments, the spherical particle has a diameter in the range from about 10 nm to about 10 μm. It is preferable that the spherical particles have a narrow particle size distribution.

In some embodiments, the spherical particles have a surface area in the range from about 10 m²/g to 1000 m²/g. In some embodiments, the porous spherical particles have a surface area in the range from about 350 m²/g to about 675 m²/g. In some embodiments, the surface area is about 350 m²/g; in other embodiments, the surface area is about 375 m²/g, in other embodiments, the surface area is about 400 m²/g; in other embodiments, the surface area is about 425 m²/g; in other embodiments, the surface area is about 450 m²/g; in other embodiments, the surface area is about 475 m²/g; in other embodiments, the surface area is about 500 m²/g; in other embodiments, the surface area is about 525 m²/g; in other embodiments, the surface area is about 550 m²/g; in other embodiments, the surface area is about 575 m²/g; in other embodiments, the surface area is about 600 m²/g; in other embodiments, the surface area is about 625 m²/g; in other embodiments, the surface area is about 650 m²/g; in still other embodiment, the surface area is about 675 m²/g; and in still other embodiments, the surface area is about 700 m²/g.

Preferably, the spherical particles used in the devices described herein are porous. In some embodiments, the spherical particles have an average pore diameter in the range from about 50 Å to about 500 Å. In some embodiments, the porosity is in the range from about 100 Å to about 400 Å, in other embodiments, the porosity is in the range from about 75 Å to about 350 Å Moreover, the average pore diameter for the spherical particles used herein may be about 50 Å, about 55 Å, about 60 Å, about 65 Å, about 70 Å, about 75 Å, about 80 Å, about 85 Å, about 90 Å, about 95 Å, about 100 Å, about 105 Å, about 110 Å, about 115 Å, about 120 Å, about 125 Å, about 150 Å, about 160 Å, about 170 Å, about 180 Å, about 190 Å, or about 200 Å.

In preferred embodiments, the polymeric binder is a biocompatible polymeric binder. Such binders include, but are not limited to polyacrylonitrile (PAN), polyethylene glycol (PEG), polypyrrole, derivatized cellulose, polysulfone, polyacrylamide, or polyamide. In a particularly preferred embodiment, binder is PAN.

In preferred embodiments, the BioSPME coating, including the microspheres and the biocompatible polymeric binder is coated on the pins by dip coating. In other embodiments, other coating methods, such as spray coating, may be used.

The coating thickness can be varied to achieve desired properties. In various embodiments, the coating thickness can be in the range from about 1 μm to about 75 μm. In preferred embodiments, the coating thickness is in the range from about 20 μm to about 50 μm. In other embodiments, the coating thickness may be, for example, about 5 μm, about 10 μm, about 15 μm about 20 μm, about 25 μm, about 30 μm, about 35 μm about 40 μm, about 45 μm, about 50 μm, about 55 μm, about 60 μm, about 65 μm, about 70 μm, or about 75 μm. In a preferred embodiment, the coating thickness is in the range from about 10 μm to about 50 μm. The coating thickness can be varied, for example, by performing the coating step multiple times. Thinner coatings, for example, may be used when sample sizes are very small, however, a thinner coating may limit the amount of analyte that may be extracted. In a preferred embodiment, the coating thickness is consistent for all pins in the upper platform.

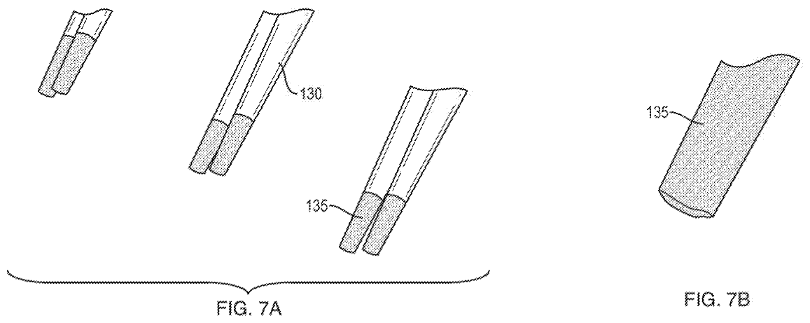

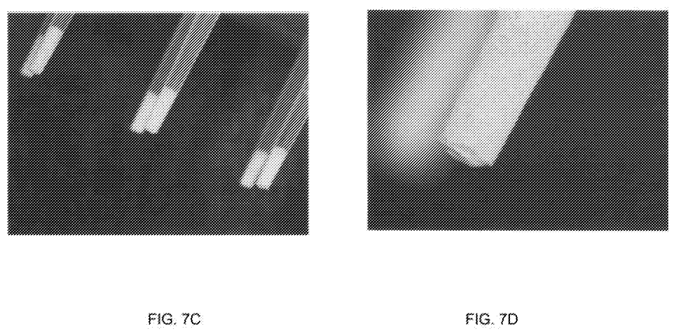

FIG. 7 illustrates the coated pins, showing the coating 135 on the tips of individual pins 130. The coated tips of the pins come into contact with the samples when the pins are disposed into the wells of the lower platform, allowing analytes of interest to be extracted. Pins coated with functionalized silica particles in a PAN binder as described herein are shown at low and high magnification in FIGS. 7C and 7D, respectively.

In other embodiments, the microspheres and the binder may be integrated into the pins when the pins are formed, such as by 3-d printing using an ink matrix that includes the binder and microspheres.

In particularly preferred embodiments, the coatings on the pins allow measurement of free and total analytes in biological samples, such as such as blood, plasma, urine, saliva, tissue, and food products. In such embodiments, the coatings absorb or adsorb certain analytes of interest, depending on the composition of the coating, while other analytes either do not interact or are physically excluded, as by size, and are thus left in solution. For example, in some embodiments, the coatings provided preferentially extract small molecule drugs, while leaving larger molecules, such as phospholipids and proteins in solution. Virtually any BioSPME coatings can be used with the apparatuses described herein allowing for the selective removal and/or concentration of various analytes of interest, or removal of interfering analytes from complex biological samples.

Some non-limiting examples of analytes that may be removed from samples using the apparatuses provided herein include small molecule drugs, such as codeine, carbamazepine, diazepam, diclofenac, fentanyl, metoprolol, propranolol, warfarin, and quinidine; illicit drugs and metabolites thereof, such as methamphetamine, cocaine and its metabolites benzoylecgonine and cocaethylene, norfentanyl, methadone and its metabolite EDDP, and the class of phenethylamine and cathinone compounds being marketed as “Bath Salts, Jewelry Cleaner or Plant Food”; as well as environmental agents, such as aldrin, atrazine, carfentrazon-ethyl, desethylatrazin, desethylterbutylazin, dichlobenil, desethylterbutylazin, dieldrin, diflufenican, endo-heptachlorepoxid, exo-heptachlorepoxid, heptachlor, linden, mefenpyr-diethyl, metolachlor, metribuzin, parathion-ethyl, parathion-methyl, pendimethalin, simazin, terbutryn, terbutylazin, and triclosan. These examples are meant to be illustrative and do not limit the devices described herein; one of ordinary skill in the art will recognize that the coatings used in the devices described herein can be adapted to extract many other analytes of interest.

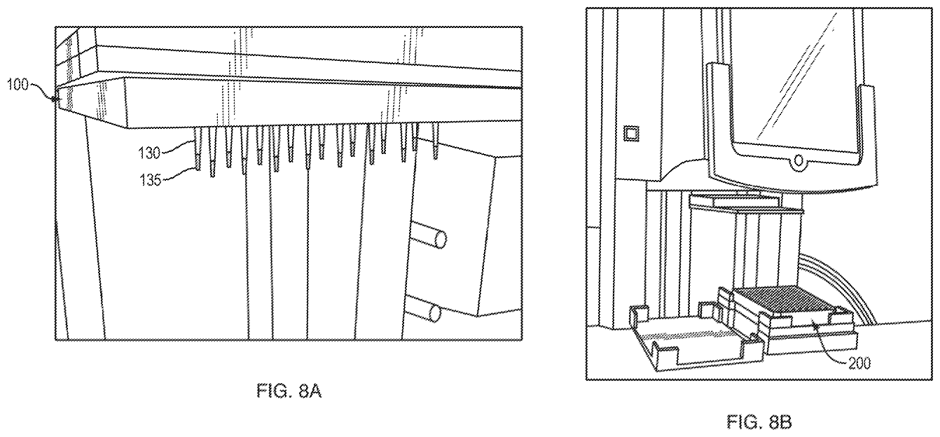

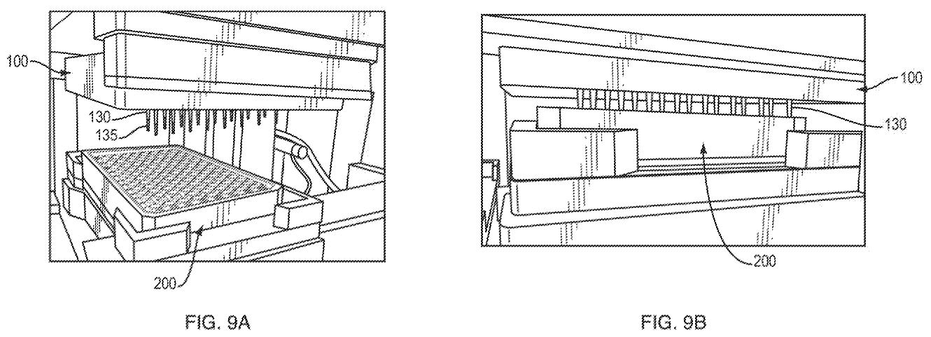

In a preferred embodiment, the apparatuses described herein are arranged such that the pins and the wells are compatible with conventional multiwell platforms, including, but not limited to 96-well platforms, 384-well platforms, and 1534-well platforms. The upper platform may be configured to have the same number of pins as the number of wells in the lower platform, or the upper platform may be configured to have a different number of pins from the number of wells in the lower platform. Because they are compatible with conventional multiwell platforms, the apparatuses described herein can be interfaced with automated sample handling systems, robotic sample management systems and instrumentation that interface with such systems. FIG. 8 shows the interface of the upper platform 100, with an automated sample handling system, pins 130, with coated tips 135 are positioned downwards such that the automated sample handling system can join the upper platform with lower platform 200 to simultaneously extract analytes from a plurality of samples. FIG. 9 shows an automated sample handling system with an apparatus described herein. In FIG. 9A, upper platform 100, with pins 130 positioned downwards is positioned above lower platform 200. In FIG. 9B, upper platform 100 has been lowered such that the pins 130 are disposed in the wells 230 of lower platform 200.

In a particularly preferred embodiment, the apparatuses described herein are adapted to interface with an automated liquid handling system and related robotic sample handling systems. In preferred embodiments, the apparatuses are adapted to interface with an automated liquid handling system and robotic sample handling system that includes an interface to a mass spectrometer. The apparatuses described herein are particularly well-suited to interface with mass spectrometers that have an ionization source such as electrospray, Desorption Electrospray Ionization (DESI), and Direct Analysis in Real Time (DART).

Also provided is a method of simultaneously isolating free analytes from a plurality of samples using the apparatuses described herein. The method is performed by adding a plurality of samples containing free analytes into the wells of the lower platform; joining the upper platform with the lower platform, wherein the pins on the upper platform are disposed in the wells of the lower platform such that the coated part of the pins is in contact with the sample; and maintaining the coated part of the pins in contact with the samples for a sufficient time for the free analytes to be extracted. The extracted analytes can then be analyzed using analytical techniques. The technique provided herein can also be used to simultaneously isolate total analytes from a plurality of samples. This can be accomplished by first extracting free analyte then adding a step to deplete bound analyte or by adjusting the exposure time to deplete the free and bound analyte.

According to the methods described herein, the pins are maintained in contact with the sample solution for a period of time sufficient to extract the analytes of interest. The extraction time will vary depending on a number of factors, including but not limited to, the nature of the sample, the SPME or BioSPME coating on the pins, the class of analytes, and so forth. In most embodiments, an extraction time in the range from about 30 seconds to about one-half hour will be sufficient to remove the analytes to the desired percentages.

In a preferred method, the apparatus coupled with an automatic liquid handling system and robotic sample handling system, and interfaced with a mass spectrometer. In such methods, the apparatus described herein can be used in the automated system to quickly and conveniently provide both the total and free analytes in the each sample.

The multi-array devices provided herein also include in vitro devices useful for direct measurement of total and free fraction analytes using an array of pins. The devices, configured in a multiplexed format compatible with conventional 96-well, 384-well and 1534-well platforms, allow for rapid analysis of samples with minimal sample volumes. Advantages of this device include the ability to automate the direct measurement of analytes within multiple samples, while enabling convenient interfacing with liquid desorption systems.

The apparatus, or device, may be configured in 8 pin, e.g., in a 1 pin-by-8 pin strip or 2 row-by-4 row plate, 12 pin; e.g. in a strip or plate configuration, 96 pin, e.g. 8 row-by-12 row plate, 384 pin, 1536 pin configurations, or combinations thereof. Moreover, the pins can be configured in any suitable arrangement. Preferably, the arrangement is one that is suitable for automated liquid handling systems and robotic sampling systems.

Because the pins include the described BioSPME coating, the device is particularly well-suited for the isolation of total and free fraction analytes from matrices using automated platforms. This multipin device allows for analysis of sample volumes between 5 μL and 5 mL. The described device is highly automatable by configuring with robotic liquid handling systems, and is easily interfaced with conventional well platform configurations. The cylindrical, rod-like, or frustoconical nature of the pins allows for high surface area exposure to the sample, thus minimizing extraction times and maximizing analyte detection. The device is highly adaptable to liquid desorption systems, along with direct mass spectrometry interfaces such as DESI, DART and other such platforms.

In a preferred embodiment, each pin has the same coating and each well may contain a different sample, allowing for quantitation of free and total analytes in many different samples. In some embodiments, however, the different pins may have different coatings, such that different pins are optimized for extracting different analytes of interest.

Use with mass spectrometry. As an SPME device, the multipin devices and methods simultaneously isolate and enrich the analytes present in a plurality of samples. Coatings used in the present disclosure may stabilize analytes that are extracted therein. Since the coating can be adjusted towards analytes of interest, devices and methods disclosed herein may reduce undesirable artifacts that might provide ion suppression or enhancement.

The coated pins may be used in various ionization methods, such as in DART (Direct Analysis in Real Time), DESI (Desorption Electrospray Ionization), OPP (Open Port Probe), SELDI (Surface Enhanced Laser Desorption Ionization), MALDI (Matrix-Assisted Laser Desorption Ionization), Liquid Extraction Surface Analysis (LESA), Liquid Microjunction Surface Sampling Probe (LMJ-SSP), or LAESI (laser ablation electrospray ionization). DART and DESI are atmospheric pressure ion source that ionizes gases, liquids and solids in open air under ambient conditions. SELDI and MALDI are soft ionization techniques that use a laser to obtain ions of the analytes. In electrospray-based devices, ions of the extracted or pre-concentrated analytes are generated directly from the edges of the solid substrate by wetting the coated solid substrate with a solvent and applying a high electric field to the wetted substrate.

As the apparatuses described herein are particularly well-suited for coupling with automated liquid sample handling systems, particularly those that interface directly with a mass spectrometer, in preferred embodiments, the apparatuses are used with laboratory benchtop mass spectrometers. However, in certain embodiment, the apparatuses described herein may be used with portable, field-deployable mass spectrometers. Representative mass analyzers include a rectilinear ion trap, a cylindrical ion trap, a quadrupole ion trap, a time-of-flight, an ion cyclotron resonance trap, and an electrostatic ion trap (for example an Orbitrap mass analyzer).

In summary, the benefits of the devices provided herein include the ability to isolate targeted analytes from difficult matrices such as biological fluids. Of particular benefit is enabling the measurement of total and free fraction of analyte within a biological sample or samples. The device, as described herein, is configured in a multiplexed format, and in preferred embodiments, is compatible with 96-well, 384-well, 1534-well, or other conventional platforms, which allows for rapid analysis of samples with minimal sample volumes. The device provides the ability to automate the direct measurement of analytes within a number of individual samples, while enabling convenient interfacing with liquid desorption, or other systems.

Coating Process

The following process is illustrative only. Coatings suitable for SPME are known to those skilled in the art. The particular coating can be chosen based on the analytes of interest. One preferred embodiment for the apparatus described herein is an SPME coating that includes silica microspheres in a PAN binder. Specific silica microsphere may be chosen based on desired properties. The polyacrylonitrile polymer can be virtually any commercially available PAN. One may choose a particular molecular weight to affect the properties of the binder. For example, a certain PAN may be chosen based on its viscosity in solution, which would, in turn, affect the coating properties when the binder/microsphere is coated on the pins as described herein.

(A) Preparation of PAN Binder Solution.

A binder solution of Polyacylonitrile (PAN) is prepared as follows. The concentration can be determined based on desired properties. In some preferred embodiments, the concentration of PAN to solvent is about 5% to about 12% (w/w). In one embodiment, the solvent is DMF, though other suitable solvents are known to those of ordinary skill in the art. Briefly, the PAN is weighed into a suitable container, preferably, the container is one that can be sealed. To the PAN, solvent is added in an amount to make the preferred concentration. The container is sealed and the suspension is shaken and/or stirred for several minutes until the PAN powder is dispersed into the solvent. The container, loosely sealed is placed in a heating block and heated, at e.g., about 80-90° C. until the PAN is completely dissolved in the solvent giving a clear solution.

(B) Preparation of a Silica Microsphere:

PAN Suspension. Silica microparticles are weighed into a container. The vial is tared an amount of the PAN solution from step (A), calculated to make the desired ratio of silica microspheres to PAN is added using a pipette. The PAN-silica suspension is mixed thoroughly until all of the silica appears wetted and dispersed in the PAN solution. The container is capped loosely and is degassed at room temperature under vacuum in a vacuum oven. After degassing, the container is removed from the oven and can be used to coat the pins.

Dip coating pin tips.

The pins integral to the upper platform are coated by dipping the tips into the PAN-silica suspension prepared in step (B). The pins are cured after the dipping application. This process is repeated until the coating is the desired thickness.

Spray coating particles on devices.

The PAN-silica suspension prepared in step (B) is placed in a spraying device suitable for spray coating the pins integral to the upper portion of the apparatus described herein. A thin layer of the particle-suspension is sprayed on the pins. The thickness of the coating suspension is controlled by the rate of application and by the number of coatings applied. The process may be accomplished by manual spaying or by using an automated coating device.

### Example

BioSPME using the coated pins.

The coated pins are used in a four step BioSPME process. The pins are conditioned by exposure to 1 mL of a 50:50 mixture of methanol and water for 20 min with 500 rpm agitation. Next, pins are placed in the wells of the bottom platform, the wells having samples including a neutral buffer solution and four analytes of interest—metoprolol, propranolol, carbamazepine, and diazepam. The pins are allowed to extract for two minutes with 500 rpm agitation. The pins are then washed with water. Finally, the pins are desorbed for 10 min within 100 μL of methanol with 500 rpm agitation. A reliable response is obtained for all four analytes, confirming their successful extraction using the silica microsphere/PAN coating described above.

The methods and example are provided for illustration only and are not meant to limit the invention claimed herein.

The invention claimed is:

1. An apparatus for simultaneously extracting one or more analytes from each of a plurality of samples, the apparatus comprising:
a housing having an upper platform and a lower platform, wherein the upper platform and the lower platform are separate pieces adapted to removably join together;
the upper platform having a top surface and a bottom surface, the bottom surface comprising a plurality of pins perpendicular to the bottom surface integrated thereto, the pins having a surface adapted to solid phase microextraction (SPME);
the lower platform having a top surface and a bottom surface, the top surface comprising a plurality of individual wells;
wherein when the upper platform and the lower platform are joined together, at least one pin of the upper platform is disposed into at least one well of the lower platform.

2. The apparatus of claim 1 wherein when the upper platform and the lower platform are joined together, each pin on the upper platform is disposed into a discrete well on the lower platform.

3. The apparatus of claim 1 wherein the pins are cylindrical or frustoconical.

4. The apparatus of claim 1 wherein the surface adapted to SPME comprises microparticles and a binder;
wherein the microparticles are selected from the group consisting of silica spheres, functionalized silica spheres, functionalized carbon spheres, polymeric resins, and combinations thereof; and
wherein the binder is a biocompatible polymer.

5. The apparatus of claim 4 wherein the surface of the pins has been coated with a coating, wherein the coating comprises functionalized silica spheres and a biocompatible binder.

6. The apparatus of claim 5 wherein the coating has a thickness in the range from about 10 μm to about 50 μm.

7. The apparatus of claim 4 wherein the biocompatible binder is selected from the group consisting of polyacrylonitrile (PAN), polyethylene glycol (PEG), polypyrrole, derivatized cellulose, polysulfone, polyacrylamide, or polyamide.

8. The apparatus of claim 7 wherein the biocompatible binder is PAN.

9. The apparatus of claim 1 wherein the pins comprise a material for SPME integral to the pins.

10. The apparatus of either one of claim 1 or 9 wherein the pins are 3-d printed.

11. The apparatus of any one of claims 1, 5, and 9 wherein the pins are adapted to measuring analytes in biological samples wherein the biological samples are selected from the group consisting of blood, plasma, urine, saliva, tissue, and food products.

12. The apparatus of claim 1 wherein the pins and the wells are compatible with conventional multiwell platforms.

13. The apparatus of claim 12 wherein the conventional multiwell platforms are selected from the group consisting of 96-well platforms, 384-well platforms, and 1534-well platforms.

14. The apparatus of claim 1 wherein the apparatus is adapted to interface with an automated liquid handling system.

15. The apparatus of claim 14 wherein the automated liquid handling system includes an interface to a mass spectrometer.

16. The apparatus of claim 15 where in the mass spectrometer includes an ionization source.

17. The apparatus of claim 16 wherein the ionization source is selected from the group consisting of electrospray, DESI, and DART.

18. A multipin device for simultaneously extracting one or more analytes from each of a plurality of sample, the device comprising:
a plate having a top surface and a bottom surface, the bottom surface comprising a plurality of pins, each pin integral to the bottom surface and projecting outwards in an approximately perpendicular manner to an end opposite the bottom surface; the pins having a surface adapted to SPME;
wherein the pins are configured to fit into a plurality of wells in a conventional multiwell platform such that the surface adapted to SPME can contact a sample disposed in one or more wells in the well plate.

19. The multipin device of claim 18 wherein the surface adapted to SPME comprises a coating, the coating comprising a plurality of functionalized silica spheres and a binder.

20. The multipin devices of claim 19 wherein the binder is a biocompatible binder.

21. The multipin device of claim 18 wherein the conventional multiwell platform is selected from a 96-well plate, a 384-well plate and a 1534-well plate.

22. A method of simultaneously extracting free analytes from a plurality of samples comprising the steps:
providing an apparatus of claim 1;
adding a plurality of samples containing free analytes into the wells of the lower platform;
joining the upper platform with the lower platform, wherein a plurality of pins on the upper platform are disposed in the samples in the wells of the lower platform such that the pins are in contact with the samples; and
maintaining the pins in contact with the samples for a sufficient time for the free analytes to be extracted.

23. A method of measuring free and total analytes in a plurality of samples comprising:
providing an apparatus of claim 1 wherein the apparatus is coupled to an automated liquid handling system and the automatic liquid handling system is coupled to a mass spectrometer;
adding a plurality of samples containing analytes to the wells of the lower platform;
joining the upper platform with the lower platform, wherein a plurality of pins on the upper platform are disposed in the samples in the wells of the lower platform such that the pins are in contact with the samples;
maintaining the pins in contact with the samples for sufficient time to extract the free analytes;
introducing the extracted free analytes to the mass spectrometer to determine the free analytes in the sample.

24. A method of simultaneously extracting free analytes from a plurality of samples comprising the steps:
providing a multipin device of claim 18 and a multiwell platform, wherein the pins of the multipin device are in a complementary configuration to the wells in the multiwell platform;
adding a plurality of samples containing free analytes into the wells of the multiwell platform;
disposing the pins of the multipin device into the wells of multiwell platform such that the pins are in contact with the samples; and
maintaining the pins in contact with the samples for a sufficient time for the free analytes to be extracted.

25. A method of measuring free and total analytes in a plurality of samples comprising:
providing a multipin device of claim 18 wherein the multipin device is coupled to an automated liquid handling system and the automatic liquid handling system is coupled to a mass spectrometer;
providing a mutliwell platform having wells in the same configuration as the pins in the multipin device wherein the multiwell platform is coupled to an automated liquid handling system;
adding a plurality of samples containing analytes to the wells of the multiwell platform;
disposing the pins of the multipin device into the well of the mulitwell platform, wherein a plurality of pins on the upper platform are disposed in the samples in the wells of the multiwell platform such that the pins are in contact with the samples;
maintaining the pins in contact with the samples for sufficient time to extract the free analytes;
introducing the extracted free analytes to the mass spectrometer to determine the free analytes in the sample.
